(19) United States
(12) Patent Application Publication
(10) Pub. No.: US 2018/0338738 A1
(43) Pub. Date: Nov. 29, 2018

(54) Method to detect acute bone trauma using sodium fluoride imaging

(71) Applicant: Osteo Diagnostics L.L.C., Tampa, FL (US)

(72) Inventor: Eric Cotton, Tampa, FL (US)

(21) Appl. No.: 15/988,510

(22) Filed: May 24, 2018

Related U.S. Application Data
(60) Provisional application No. 62/510,933, filed on May 25, 2017.

(51) Int. Cl. A61B 6/00; A61B 6/03; A61M 5/00; A61K 51/02

(52) U.S. Cl. CPC A61B 6/505; A61B 6/032; A61B 6/037; A61K 51/02; A61B 6/5235; A61M 5/007; A61B 6/5217

(57) ABSTRACT

Minor injuries and acute bone traumas are difficult to detect with current methods of bone imaging. The use of ¹⁸F—NaF in combination with PET/CT provides superior imaging of bone trauma, particularly for traumas are difficult to detect using known methods. The superior uptake of ¹⁸F—NaF, as compared to existing chemicals, allows for detection of areas of acute bone trauma that may not have presented any pain symptoms, and thus using other methods would avoid diagnosis.

### CROSS-REFERENCE TO RELATED APPLICATION

This application is a non-provisional application that claims priority to U.S. provisional application No. 62/510,933, filed May 25, 2017.

### FIELD

This invention relates to the field of Positron-Emission Tomography (PET) and Computed Tomography (CT) scanning and more particularly to a system for detecting acute bone trauma from car accidents using ¹⁸F-sodium fluoride.

### BACKGROUND

Automobile accidents can have a devastating effect on the human body. Even at low speeds, accidents sometimes cause serious injuries to vehicle occupants. Despite the advances in vehicle safety, such as airbags, seat belts, and bumpers, most car crashes result in some injury to the occupants.

Broken bones and lacerations are straightforward to detect and treat. But minor injuries may evade detection, potentially not causing pain until weeks or months after the initial trauma. These minor, undetected injuries may worsen as the body tries to heal them without medical intervention. Such worsening injuries may be avoided if detected early, and an appropriate treatment prescribed and performed.

What is needed is a method to detect acute bone traumas caused by automobile accidents. To detect minor injuries the method needs an improved uptake and contrast as compared to standard bone scintigraphy methods.

### SUMMARY

Disclosed within is a methodology for the use of ¹⁸F-sodium fluoride (¹⁸F—NaF), in combination with a PET/CT or PET/MRI scanner for the detection of bone trauma resulting from accidents. As compared to existing methods, the method disclosed has increased sensitivity and therefore superior bone damage detection.

First, a discussion of the scanning technology.

PET/CT scanning is a molecular imaging technology that combines cross-sectional, functional, and anatomic imaging for diagnosis. PET/CT machines combine a PET scanner and a CT scanner into a single device.

Within the combined PET/CT machine, the PET scanner detects the emissions of a radioactive molecule. The CT scanner uses a computer to process and combine multiple x-ray images taken from different perspectives. The resulting images from the PET scan and CT scan are superposed to create a single image, thus combining the benefits of each type of scanning.

The PET is optionally combined with Magnet Resonance Imaging (MRI) in place of a CT scanner. The resulting readings are more useful for certain types of diagnosis.

PET/CT scanning may be limited to a single anatomic region, such as the head and neck, thorax, or abdomen and pelvis. PET/CT scanning may be limited to the portion of the body between the skull base and middle of the thighs or may be used to create images of the entire body from the head to the toes.

Second, a review of the imaging agent, ¹⁸F-fluouride, administered as intravenous Na¹⁸F.

¹⁸F-fluouride is a highly sensitive, bone-seeking PET tracer used for detection of skeletal abnormalities. ¹⁸F-fluouride is a diagnostic molecular imaging agent used for identification of new bone formation.

¹⁸F-fluouride is quickly cleared from plasma with most of the tracer retained by bone after a single pass. The tracer uptake by the bone is the result of chemisorption with exchange of ¹⁸F⁻ ion for OH⁻ ion on the surface of the hydroxyapatite matrix of bone. The result is the formation of fluoroapatite and migration of ¹⁸F⁻ ion into the crystalline matrix of bone. The minimal binding to serum protein and rapid renal clearance contribute to the high quality of images with high bone-to-background ratio in a shorter time than for standard ^{99m}Tc-based tracers. ¹⁸F-fluouride may be used with dynamic PET data acquisition to obtain quantitative measurements of the tracer pharmacokinetics. The measurements are useful in clinical and research applications.

A key clinical application of ¹⁸F-fluouride is the detection of acute bone trauma resulting from automobile accidents. During major automobile accidents with severe trauma, a person or doctor can easily identify areas that require treatment or monitoring to heal. But in less severe accidents, the vehicle occupants may have internal injuries that are hard to detect using conventional diagnostic methods.

The disclosed method is particularly effective for patients with suspected sternal injuries, rib injuries, and spine injuries.

More generally, patients with pre-existing orthopedic hardware benefit from a sensitive diagnostic method that does not require the use of magnetic fields, i.e., MRI. Common orthopedic hardware includes shoulder prostheses, hip prostheses, knee prostheses, and implantable orthopedic rods.

The timeline of application of the disclosed method influences its effectiveness. Ideally, the method is applied within six months of the initial trauma. But, the method is useful for as long as two years after the initial trauma.

The benefit of ¹⁸F—NaF is its strong uptake in areas of bone with minimal trauma. Thus, a small fracture or even a bone bruise is detected using the ¹⁸F—NaF injection and subsequent PET/CT scan.

Other uses of ¹⁸F—NaF include detecting trauma associated with: benign bone and joint diseases; child abuse; osteomyelitis; arthritis; avascular necrosis; metabolic bone disease; Paget disease; complications of prosthetic joints; reflex sympathetic dystrophy; and malignant diseases such as cancers.

### DESCRIPTION OF THE DRAWINGS

FIG. 1 is a flowchart of the steps required to perform the method.

### DETAILED DESCRIPTION

Reference will now be made in detail to the presently preferred embodiments of the invention.

The disclosed method is performed as follows:
First—Intake

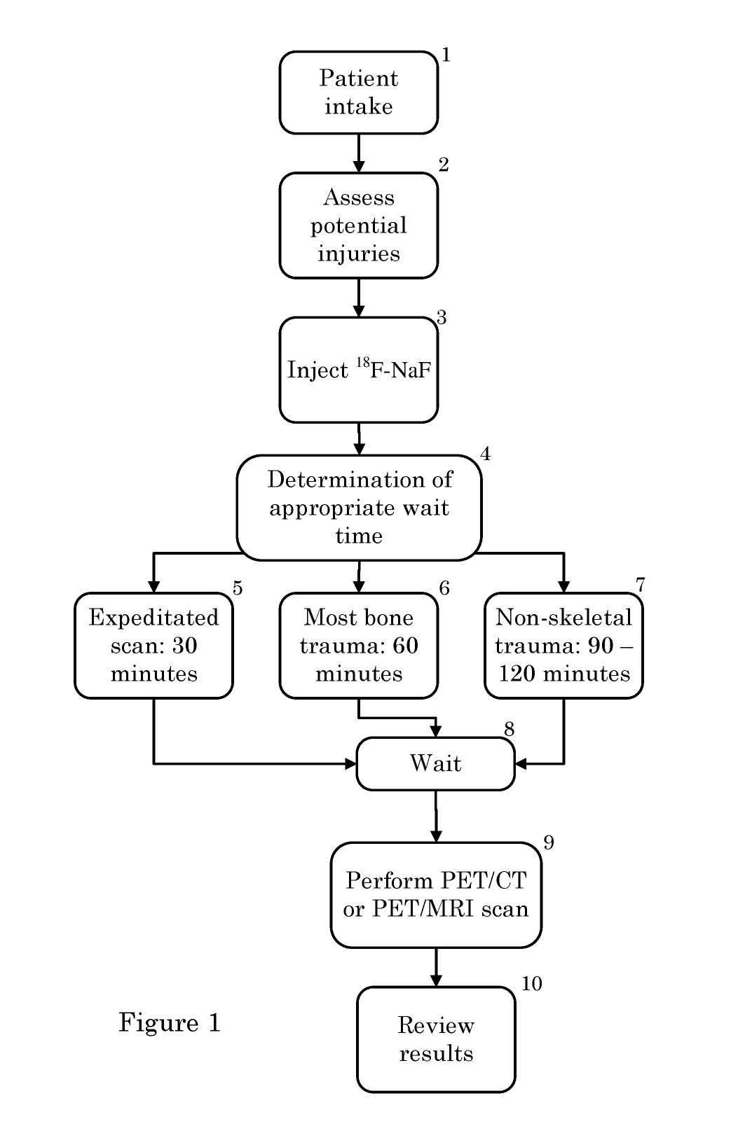

The patient is checked into the scanning facility, and the height and weight of the patient are measured and recorded. Shown as patient intake 1 in FIG. 1.

The patient is interviewed to determine the circumstances of the incident, including date, time, and type. Further recorded are initial symptoms, current symptoms, and whether any prior imaging has been performed. Shown as assess potential injuries 2 in FIG. 1.
Second—Injection of ¹⁸F—NaF

The patient is connected to an intravenous (IV) bag via an IV line. Shown as inject ¹⁸F—NaF in FIG. 1.

Ten mCi of ¹⁸F—NaF is drawn and assayed with the dose calibrator. A Ci, or Curie, is a non-SI unit of radioactivity. One curie (1 Ci) is equal to 3.7×10¹⁰ radioactive decays per second. This is approximately equal to the number of decays that occur in one gram of radium per second.

The becquerel is an SI unit of radioactivity, abbreviated as Bq. The conversion is as follows=1 Ci=3.7×10¹⁰ Bq.

The drawn dosage is then injected into the subject's IV line. The time of injection is recorded.

The IV line is flushed with saline.

The syringe is assayed, and the quantity of residual radioactivity within the syringe is recorded.

The correct amount of wait time must then be determined. Shown as determination of appropriate wait time 4 in FIG. 1.

PET images of the axial skeleton may begin as soon as 30-45 min after administration of the radiopharmaceutical in patients with normal renal function, because of the rapid localization of ¹⁸F in the skeleton and rapid clearance from the circulation. Shown as expedited scan 5 in FIG. 1.

In embodiments looking at non-skeletal trauma, it is necessary to wait longer to obtain high-quality images of the extremities. For these embodiments, a wait time of 90-120 min is preferred. Shown as non-skeletal trauma 7 in FIG. 1.

For the method disclosed within, the preferred time wait time is sixty minutes. Shown as most bone trauma 6 in FIG. 1.

The patient is asked to rest within a quiet room held at a comfortable temperature and wait a set amount of time. Shown as wait 8 in FIG. 1.

As the patient is guided to the PET/CT or PET/MRI scanner, the patient uses the restroom and empties his or her bladder.

The patient is placed supine, or face-up, in the PET/CT or PET/MRI scanner. The patient is placed with arms up.

The scanner is energized and proceeds to scan the desired location or locations. Scan coverage includes the entire body from the top of the head to the bottom of the feet. Shown as perform PET/CT or PET/MRI scan 9 in FIG. 1.

The following scan settings are used:
Tube voltage is 120 kVp, and tube current is 45 mAs. The unit kVp, or Kilovoltage peak, is the peak voltage applied to the x-ray tube. The kVp determines the highest energy of an x-ray photon. The unit mAs, or milliamperage-seconds, is the product of milliamps and seconds.
Scan filler view is large.
Slice thickness is 3.0 mm with no gap.
Scan time is 3 minutes per bed position.

After the scan is complete, edge data is reviewed for quality assurance.

After quality assurance has been performed, the image data is sent to a dedicated molecular imaging workstation for review.

As an alternative, because of the high bone to soft-tissue activity ratio of ¹⁸F bone scans, high-quality images may be obtained without requiring a CT scan for attenuation correction. Thus, it is possible to survey the whole body with emission-only images and then acquire additional images, as needed, using PET/CT of a limited area.
Third—Imaging Review

Imaging review is best performed by a radiologist who is board certified in nuclear medicine. Shown as review results 10 in FIG. 1.

The patient's clinical history and pertinent prior imaging studies and reports are reviewed prior to reviewing the image data.

The PET/CT or PET/MRI fused data sets are reviewed to assess for any detectable regions of abnormal sodium fluoride accumulation within the skeleton.

¹⁸F is normally distributed throughout the entire skeleton. The major route of excretion is the urinary tract. The patient's kidneys, ureters, and bladder should be visible in the absence of renal insufficiency. The degree of localization in the urinary tract depends on renal function, state of hydration, and interval between administration of 18F and imaging. Renal insufficiency will decrease localization in the urinary tract. Urinary outflow obstruction will increase localization proximal to the site of obstruction. Chronic severe obstruction, however, may reduce localization. Soft-tissue activity reflects the amount of circulating ¹⁸F in the blood pool at the time of imaging and should be minimal. Local or regional hyperemia may cause increased visualization of the soft tissues.

¹⁸F localization in the skeleton is dependent on regional blood flow, as well as on new bone formation. ¹⁸F is substituted for hydroxyl groups in hydroxyapatite and covalently bonds to the surface of new bone. Uptake is higher in new bone (osteoid) because of higher availability of binding sites. Local or regional hyperemia may also cause increased localization in the skeleton.

Physiologic ¹⁸F uptake in the skeleton is generally uniform in adults. Normal growth causes increased localization in the metaphyses—the wide section at the end of the shaft of a long bone—of children and adolescents. Symmetric uptake between the left and right sides is generally observed in individuals of all ages, except in periarticular sites, where ¹⁸F uptake can be variable.

Nearly all causes of increased new-bone formation cause increased localization of ¹⁸F. The degree of increased localization is dependent on many factors, including blood flow and the amount of new bone formation. Processes that result in minimal osteoblastic activity, or primarily osteolytic activity, may not be detected.

In general, the degree of ¹⁸F uptake does not differentiate benign from malignant processes. The pattern of ¹⁸F uptake, however, may be suggestive or even characteristic of a specific diagnosis. Correlation with skeletal radiographs and other anatomic imaging is essential for diagnosis. The CT component of PET/CT, even when performed primarily for attenuation correction and anatomic registration, also provides diagnostic information.

Any degree of ¹⁸F uptake that is visibly higher or lower than uptake in adjacent bone, or uptake in the corresponding contralateral region, indicates an alteration in bone metabolism. Because of the higher resolution of PET/CT, compared with single-photon imaging, physiologic variability is more prominent. The increased uptake directly correlates to sites of acute bone trauma. This makes this method of ¹⁸F in combination with a PET scanner to be excellent for diagnosing automobile accidents.

Regions of abnormal sodium fluoride uptake within the skeleton can be sampled for quantitative SUV (Standard Uptake Value) analysis.

Areas of abnormal sodium fluoride uptake on the PET and PET/CT or PET/MRI data sets are carefully correlated with the corresponding CT data set. The CT data set is carefully reviewed in the areas of abnormal sodium fluoride uptake to determine if there is anatomic evidence for an underling bone injury such as fracture or callus formation.

After careful review of the PET, PET/CT or PET/MRI CT data sets, a report is generated delineating any abnormalities within the axial or appendicular skeleton.

The report is generated and sent to the referring physician.

Equivalent elements can be substituted for the ones set forth above such that they perform in substantially the same manner in substantially the same way for achieving substantially the same result.

It is believed that the system and method as described and many of its attendant advantages will be understood by the foregoing description. It is also believed that it will be apparent that various changes may be made in the form, construction, and arrangement of the components thereof without departing from the scope and spirit of the invention or without sacrificing all of its material advantages. The form herein before described being merely exemplary and explanatory embodiment thereof. It is the intention of the following claims to encompass and include such changes.

What is claimed is:

1. A method of detecting trauma to one or more bones, the trauma caused by an automobile accident, the method comprising:
injecting a patient with ¹⁸F—NaF, the ¹⁸F—NaF separating to create ¹⁸F ions;
waiting a period of time to allow the ¹⁸F ions of the ¹⁸F—NaF to interact with the one or more bones;
the ¹⁸F ion predominantly interacting with the one or more bones in locations of growth or repair;
analyzing the uptake of ¹⁸F—NaF in patient's bones using a PET/CT scanner; and
reviewing the combined PET and CT images to locate bone trauma.

2. The method of claim 1, wherein the ¹⁸F—NaF has a radioactive value of ten mCi.

3. The method of claim 1, wherein the period of time is thirty minutes, which is the minimum time required for the ¹⁸F ions to accumulate in damaged sections of the one or more bones.

4. The method of claim 1, wherein the period of time is sixty minutes, an average time for use in most applications for sufficient accumulation of ¹⁸F ions within damaged sections of the one or more bones.

5. The method of claim 1, wherein the period of time is one hundred and twenty minutes, a period of time to highlight more subtle damage by the accumulation of ¹⁸F ions within damaged sections of the one or more bones.

6. A method of detecting trauma to one or more bones, the trauma caused by an automobile accident, the method comprising:
injecting a patient with ¹⁸F—NaF, the ¹⁸F—NaF separating to create ¹⁸F ions;
waiting a period of time to allow the ¹⁸F ions of the ¹⁸F—NaF to interact with the one or more bones;
the ¹⁸F ion predominantly interacting with the one or more bones in locations of growth or repair;
analyzing the uptake of ¹⁸F—NaF in patient's bones using a PET/CT scanner, the PET/CT scanner set as follows:
tube voltage of 120 kVp;
tube current of 45 mAs;
Scan filler view set at large;
Slice thickness set at 3.0 mm with no gap;
Scan time set at 3 minutes per bed position;
reviewing the combined PET and CT images to locate bone trauma.

7. The method of claim 6, wherein the ¹⁸F—NaF has a radioactive value of ten mCi.

8. The method of claim 6, wherein the period of time is thirty minutes, which is the minimum time required for the ¹⁸F ions to accumulate in damaged sections of the one or more bones.

9. The method of claim 6, wherein the period of time is sixty minutes, an average time for use in most applications for sufficient accumulation of ¹⁸F ions within damaged sections of the one or more bones.

10. The method of claim 6, wherein the period of time is one hundred and twenty minutes, a period of time to highlight more subtle damage by the accumulation of ¹⁸F ions within damaged sections of the one or more bones.

11. A method of detecting trauma to one or more bones, the trauma caused by an automobile accident, the method comprising:
injecting a patient with ten mCi of ¹⁸F—NaF, the ¹⁸F—NaF separating to create ¹⁸F ions;
waiting a period of time to allow the ¹⁸F ions of the ¹⁸F—NaF to interact with the one or more bones;
the ¹⁸F ion predominantly interacting with the one or more bones in locations of growth or repair;
analyzing the uptake of ¹⁸F—NaF in patient's bones using a PET/MRI scanner; and
reviewing the combined PET and MRI images to locate bone trauma.

12. The method of claim 10, wherein the period of time is thirty minutes, which is the minimum time required for the ¹⁸F ions to accumulate in damaged sections of the one or more bones.

13. The method of claim 10, wherein the period of time is sixty minutes, an average time for use in most applications for sufficient accumulation of ¹⁸F ions within damaged sections of the one or more bones.

14. The method of claim 10, wherein the period of time is one hundred and twenty minutes, a period of time to highlight more subtle damage by the accumulation of ¹⁸F ions within damaged sections of the one or more bones.
